(19) United States
(12) Patent Application Publication
(10) Pub. No.: US 2024/0226070 A1
(43) Pub. Date: Jul. 11, 2024

(54) URAT1 INHIBITOR, PHARMACEUTICAL COMPOSITIONS AND USES THEREOF

(71) Applicant: UR-1 Therapeutics, Inc., New York, NY (US)

(72) Inventors: Lindsay Rosenwald, New York, NY (US); Lei Zheng, New York, NY (US)

(21) Appl. No.: 18/285,841

(22) PCT Filed: Apr. 6, 2022
(86) PCT No.: PCT/US2022/023730

§ 371 Date: Oct. 5, 2023

Related U.S. Application Data
(60) Provisional application No. 63/172,440, filed on Apr. 8, 2021.
(60) Provisional application No. 63/171,774, filed on Apr. 7, 2021.

(51) Int. Cl. A61K 31/426; A61K 31/519; A61K 38/44; A61K 45/06; A61P 13/12

(52) U.S. Cl. CPC A61K 31/426; A61K 31/519; A61K 38/44; A61K 45/06; A61P 13/12; C12Y 107/03003

(57) ABSTRACT

Provided herein are pharmaceutical compositions including a URAT1 inhibitor and methods of use thereof. The pharmaceutical compositions can include dotinurad, a xanthine oxidase inhibitor, such as allopurinol and/or a sodium-glucose cotransporter-2 inhibitor. The pharmaceutical compositions described herein can be used for the treatment of diseases and conditions related to uric acid, including chronic kidney disease.

### CROSS-REFERENCE TO RELATED APPLICATIONS

This application claims the benefit of priority under 35 U.S.C. § 119(e) of U.S. Provisional Patent Application Nos. 63/171,774 filed Apr. 7, 2021 and 63/172,440 filed Apr. 8, 2021, the entire contents of each application is incorporated herein by reference in their entirety.

### BACKGROUND OF THE INVENTION

### Field of the Invention

The present invention relates generally to pharmaceutical compositions including a URAT1 inhibitor and more specifically to uses thereof for the treatment of chronic kidney disease (CKD), non-alcoholic steatohepatitis (NASH), heart failure, gout and/or hyperuricemia and other related indications.

### Background Information

Uric acid is a heterocyclic compound of carbon, nitrogen, oxygen, and hydrogen with the formula C₅H₄N₄O₃. It forms ions and salts known as urates and acid urates, such as ammonium acid urate. Uric acid is a product of the metabolic breakdown of purine nucleotides, and it is a normal component of urine. High blood concentrations of uric acid can lead to gout and are associated with other medical conditions, including diabetes and the formation of ammonium acid urate kidney stones. Xanthine oxidase is an enzyme which catalyzes the formation of uric acid from xanthine and hypoxanthine, which in turn are produced from other purines. Xanthine oxidase is a large enzyme whose active site consists of the metal molybdenum bound to sulfur and oxygen. Within cells, xanthine oxidase can exist as xanthine dehydrogenase and xanthine oxidoreductase.

The normal concentration range of uric acid (or hydrogen urate ion) in human blood is 25 to 80 mg/L for men and 15 to 60 mg/L for women. In humans, about 70% of daily uric acid disposal occurs via the kidneys, and in 5-25% of humans, impaired renal (kidney) excretion leads to hyperuricemia. Normal excretion of uric acid in the urine is 250 to 750 mg per day (concentration of 250 to 750 mg/L). In human blood plasma, the reference range of uric acid is typically 3.4-7.2 mg per 100 ml (200-430 μmol/L) for men, and 2.4-6.1 mg per 100 ml for women (140-360 μmol/L). Uric acid concentrations in blood plasma above and below the normal range are known as, respectively, hyperuricemia and hypouricemia. Likewise, uric acid concentrations in urine above and below normal are known as hyperuricosuria and hypouricosuria. Uric acid levels in saliva may be associated with blood uric acid levels. Hyperuricemia (high levels of uric acid), which induces gout, has various potential origins including diet (high intake of dietary purine, high-fructose corn syrup, and table sugar can increase levels of uric acid), reduced excretion via the kidneys, fasting or rapid weight loss, certain drugs (such as thiazide diuretics can increase blood uric acid levels by interfering with renal clearance), and tumor lysis syndrome due to nucleobase and potassium release into the plasma).

Hyperuricemia has been associated with chronic kidney disease and renal dysfunction and identified as an independent risk factor for renal function decline. Hyperuricemia has also been associated with cardiovascular disease and heart failure. Chronic kidney disease is a leading cause of mortality and disability worldwide. The disease progressive nature is independent of the initial insult and leads to scarring and renal function loss. Importantly, cardiovascular (CV) complications are common in patients with chronic kidney disease (CKD) as alterations in phosphorus and calcium metabolism predispose to develop aortic vascular calcification in such patients. Hyperuricemia is a common alteration in CKD. Several clinical and experimental studies have suggested a potential causal role of uric acid in renal disease, and the topic is still under hot debate. Moreover, urate-lowering therapy (ULT) reduces the progression of CKD and decreases the relative risk for developing CV events in CKD patients as hyperuricemia, even asymptomatic, is associated with coronary calcification. The pathophysiological mechanisms associated with these deleterious effects currently are not well understood.

Thus, a need exists for therapeutic methods and compositions for reducing serum uric acid levels that may be used in therapeutic and prophylactic methods, e.g. to treat or prevent conditions associated with hyperuricemia such as chronic kidney disease and heart failure.

### SUMMARY OF THE INVENTION

The present invention is based on the seminal discovery that a pharmaceutical composition including a URAT1 inhibitor alone or in combination with other agents, such as a xanthine oxidase (XO) inhibitor and/or a sodium-glucose cotransporter-2 (SGLT2) inhibitor can be used for the treatment of various diseases and conditions including chronic kidney disease (CKD), non-alcoholic steatohepatitis (NASH), heart failure, gout and/or hyperuricemia and other related indications.

In one embodiment, the present invention provides a pharmaceutical composition including a URAT1 inhibitor alone or in combination with a sodium-glucose cotransporter-2 (SGLT2) inhibitor and/or a xanthine oxidase (XO) inhibitor and a pharmaceutical carrier.

In one aspect, the composition is for the treatment of chronic kidney disease (CKD). In another aspect, the URAT1 inhibitor is dotinurad, a pharmaceutically acceptable salt, hydrate, or solvate thereof. In other aspects, dotinurad is an amorphous form. In one aspect, dotinurad is a type I crystal having characteristic peaks at least about 11.5, 14.6, 18.2, 24.0 and 25.5 degrees in a diffraction angle (2θ) by X-ray powder diffraction. In many aspects, the type I dotinurad crystal has a heat absorption peak at about 191° C. in DSC analysis. In one aspect, dotinurad is a type II crystal having characteristic peaks at least about 15.1, 18.1, 22.8, 23.7 and 24.0 degrees in a diffraction angle (2θ) by X-ray powder diffraction. In many aspects, the type II dotinurad crystal has a heat absorption peak around 212° C. in differential scanning calorimetry (DSC) analysis. In one aspect, the composition is formulated in an oral dosage form. In various aspects, the oral dosage form is a tablet or a capsule. In some aspects, the tablet or capsule includes 0.1-20 mg of dotinurad. In one aspect, the SGLT2 inhibitor is selected from canagliflozin, dapagliflozin, empagliflozin, ertugliflozin, ipraglifolozin, luseogliflozin, remoglifolozin, sergliflozin, sotagliflozin, or tofogliflozin. In other aspects, the XO inhibitor is selected from allopurinol, oxypurinol, tisopurine, febuxostat, topiroxostat or an inositol. In some aspects, the XO inhibitor is allopurinol or febuxostat. In one aspect, allopurinol is present at 100-800 mg. In another aspect, febuxostat is present at 10-200 mg. In one aspect, the composition is formulated in a fixed dose combination.

In another embodiment, the invention provides a method of treating chronic kidney disease (CKD) in a subject including administering to the subject a therapeutically effective amount of any of the pharmaceutical compositions described herein or a composition including a URAT1 inhibitor.

In various aspects, the URAT1 inhibitor is dotinurad. In one aspect, the CKD is characterized by albuminuria, proteinuria, elevated creatinine clearance, hyperuricemia, inflammatory infiltration, tubular damage, fibrosis, reduced glomerular filtration rate, high blood pressure, type 2 diabetes, or a combination thereof.

In an additional embodiment, the invention provides a method of treating non-alcoholic fatty liver disease (NAFLD) and/or non-alcoholic steatohepatitis (NASH) in a subject including administering to the subject a therapeutically effective amount of any of the pharmaceutical compositions described herein or a composition including a URAT1 inhibitor.

In a further embodiment, the invention provides a method of treating heart failure in a subject including administering to the subject a therapeutically effective amount of any of the pharmaceutical compositions described herein or a composition including a URAT1 inhibitor.

In one embodiment, the present invention provides a method of treating gout and/or hyperuricemia in a subject including administering to the subject a therapeutically effective amount of any of the pharmaceutical compositions described herein or a composition including a URAT1 inhibitor.

In one aspect, an effective amount of pegloticase is further administered to the subject.

### DETAILED DESCRIPTION OF THE INVENTION

The present invention is based on the seminal discovery that a pharmaceutical composition including dotinurad alone or in combination with another agent, such as a xanthine oxidase (XO) inhibitor and/or a sodium-glucose cotransporter-2 (SGLT2) inhibitor can be used for the treatment of various diseases and conditions including chronic kidney disease (CKD), non-alcoholic steatohepatitis (NASH), heart failure, gout and/or hyperuricemia and other related indications.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference. Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a pharmaceutical composition including a URAT1 inhibitor alone or in combination with a sodium-glucose cotransporter-2 (SGLT2) inhibitor and/or a xanthine oxidase (XO) inhibitor and a pharmaceutical carrier.

Solute carrier family 22 (organic anion/cation transporter), member 12, also known as SLC22A12 and URAT1, is a urate transporter and urate-anion exchanger which in humans is encoded by the SLC22A12 gene. URAT1 is an integral membrane protein primarily found in kidney which regulates the level of urate in the blood.

URAT1 is a member of the OAT (organic anion transporter) family and was first cloned from the human kidney, where it is localized to the apical (brush border) membrane of renal proximal tubular cells. URAT1 mediates the reabsorption of uric acid, thereby regulating blood uric acid concentrations. Impairment in URAT1 activity, either due to polymorphisms, or drug-drug interactions, can have toxicological consequences. In the kidney, URAT1 is distributed along the renal tubular cell membrane and involved in reabsorption and excretion of uric acid, organic acids, drugs and their metabolites. Uric acid is taken up by OAT1 and OAT3 from the blood and reabsorbed into renal tubular cells via URAT1, in exchange for dicarboxylic acid. URAT1, along with OAT4 mediates uptake of uric acid from the renal tubule into renal tubular cells in exchange for organic anions such as lactic acid and nicotinic acid. This exchange is electroneutral and can be trans-stimulated by Cl− gradients and gradients of lactate transported by the sodium-monocarboxylate transporter.

Levels of urate anion in the blood are regulated in part by urate transporters. In particular instances, the urate transporter is URAT1. In some instances, single nucleotide polymorphisms of the gene which expresses URAT1 are significantly associated with increased or decreased reabsorption of uric acid by the kidneys, which contributes to hyperuricemia and hypouricemia, respectively. Disclosed herein is the use of the URAT1 inhibitor dotinurad in chronic kidney disease (CKD) combination therapy. The URAT1 inhibitor is dotinurad, or a pharmaceutically acceptable salt thereof.

In one aspect, the URAT1 inhibitor is dotinurad, a pharmaceutically acceptable salt, hydrate, or solvate thereof.

Dotinurad is a potent and selective urate reabsorption inhibitor that inhibits urate transporter 1 (URAT1) with an IC50 value of 37.2 nM. Dotinurad acts as a uricosuric agent. In vitro, Dotinurad has a higher selectivity for urate transporter 1 (URAT1) versus ATP-binding cassette subfamily G member 2 (ABCG2) and OAT 1/3. Dotinurad weakly inhibits ABCG2, OAT1, and OAT3, with IC50 values of 4.16, 4.08, and 1.32 μM, respectively. In vivo, Dotinurad exhibits low oral bioavailability (2.5%) and Cmax (415 ng/mL) following oral administration (1.3 mg/kg). Dotinurad exhibits terminal elimination half-life (T1/2 1.88 h) due to high plasma clearance (24126 mL/h/kg) following oral administration (1.3 mg/kg) in Sprague-Dawley rats.

The compound dotinurad, with a generic name of 3-(3,5-dichloro-4-hydroxybenzoyl)-1,1-dioxo-2,3-dihydro-1,3-benzothiazole, and a CAS number of 1285572-51-1, has the following chemical structure:

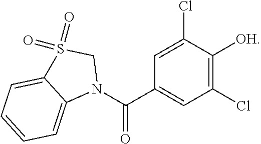 [SMILES: O=C(c1cc(Cl)c(O)c(Cl)c1)N1CS(=O)(=O)c2ccccc21]

Dotinurad can be synthesized according to U.S. Pat. No. 8,367,843, which is incorporated herein by reference in its entirety. In some aspects, dotinurad is an amorphous form. The salt, crystal, hydrate, or solvate forms of dotinurad can be synthesized according to U.S. Pat. No. 10,752,601, which is incorporated herein by reference in its entirety.

In some aspects, a pharmaceutically acceptable salt of dotinurad can be a sodium salt of dotinurad, i.e. sodium 3-(3,5-dichloro-4-hydroxybenzoyl)-1,1-dioxo-2,3-dihydro-1,3-benzothiazole, with a CAS number of 2249800-50-6.

In other aspects, the dotinurad is a 1:1 hydrate, i.e. 3-(3,5-dichloro-4-hydroxybenzoyl)-1,1-dioxo-2,3-dihydro-1,3-benzothiazole hydrate, with a CAS number of 2249800-49-3. In some aspects, the dotinurad hydrate has characteristic peaks at about 9.5, 13.7. 22.8, 24.9, and 25.3 degrees in a diffraction angle (20) by X-ray powder diffraction. In certain aspects, the dotinurad hydrate has heat absorption peaks at about 105° C. and 212° C. in DSC analysis.

In certain aspects, dotinurad can be a crystalline solvate with 1,2-dimethoxyethane. In some aspects, the dotinurad/1,2-dimethoxyethane crystalline solvate can have characteristic peaks at about 5.86, 11.98, 20.7, 24.1, and 25.5 in a diffraction angle (20) by X-ray powder diffraction.

In some aspects, dotinurad can be a crystalline solvate with 1,4-dioxane. In other aspects, the dotinurad/1,4-dioxane crystalline solvate can have characteristic peaks at about 8.14, 12.78, 22.54, 24.22, and 25.02 in a diffraction angle (20) by X-ray powder diffraction. In certain aspects, the dotinurad/1,4-dioxane crystalline solvate has heat absorption peaks at about 153° C., 186° C., and 212° C. in DSC analysis.

In many aspects, dotinurad can be a crystalline solvate with acetonitrile. In other aspects, the dotinurad/acetonitrile crystalline solvate can have characteristic peaks at about 7.86, 12.62, 22.54, 24.3, and 32.82 in a diffraction angle (20) by X-ray powder diffraction.

In some aspects, dotinurad can be a 1:1 crystalline solvate with 2-butanone, with a CAS number of 2249800-56-2.

In other aspects, dotinurad can be an acetone/isopropyl ether solvate of dotinurad having characteristic peaks at about 7.06, 12.22, 21.66, 23.5, and 24.5 in a diffraction angle (2θ) by X-ray powder diffraction. In certain aspects, the acetone/isopropyl ether solvate of dotinurad has heat absorption peaks at about 88° C. and 212° C. in DSC.

In some aspects, dotinurad can be a methyltetrahydrofuran solvate of dotinurad having characteristic peaks at about 9.54, 16.74, 21.02, 22.94, and 26.38 in a diffraction angle (2θ) by X-ray powder diffraction. In certain aspects, the methyltetrahydrofuran solvate of dotinurad has heat absorption peaks at about 97.6° C. and 212° C. in DSC.

In other aspects, dotinurad can be a 2-butanone/isopropanol solvate of dotinurad having characteristic peaks at about 7.86, 12.62, 22.54, 24.3, and 32.82 in a diffraction angle (2θ) by X-ray powder diffraction.

In some aspects, dotinurad can be a crystalline solvate with tetrahydrofuran. In other aspects, dotinurad can be a type I crystal having characteristic peaks at least about 11.5, 14.6, 18.2, 24.0 and 25.5 degrees in a diffraction angle (20) by X-ray powder diffraction. In certain aspects, the type I dotinurad crystal has a heat absorption peak at about 191° C. in DSC analysis.

As a non-limiting example to obtain type I dotinurad crystal, 40 mL tetrahydrofuran was used to dissolve 10.0 g of dotinurad. 10 mg seed crystals were added to 200 mL 2-propanol. The obtained mixture was cooled to −25° C. and stirred, and a solution of dotinurad in tetrahydrofuran was added dropwise over 15 minutes. The crystals precipitated at 0° C. were collected by filtration and washed with 20 mL propanol and dried overnight at 80° C. in vacuo to obtain type I crystal.

In some aspects, dotinurad is a crystalline solvate formed with ethyl acetate and 2-propanol. In various aspects, dotinurad is a type II crystal having characteristic peaks at least about 15.1, 18.1, 22.8, 23.7 and 24.0 degrees in a diffraction angle (20) by X-ray powder diffraction. In many aspects, the type II dotinurad crystal has a heat absorption peak at about 212° C. in differential scanning calorimetry (DSC) analysis.

As a non-limiting example to obtain type II dotinurad crystal, 45 mL ethyl acetate and 285 mL 2-propanol were used to dissolve 15.0 g dotinurad. The solution was cooled to about 25° C., the precipitated crystals were collected by filtration and washed with 20 mL 2-propanol and dried overnight at 100° C. in vacuo to obtain type II crystal.

According to some aspects of the present invention, the X-ray powder diffraction spectrum can be measured using MiniFlex (Rigaku Corporation) under the following conditions:
X-ray source: Cu,
Goniometer: vertical type,
Divergence slit: variable,
Scattering slit: 4.2 deg,
Receiving slit: 0.3 mm,
Scanning mode: continuous,
Scanning speed: 2/min,
Sampling width: 0.01°
Scan axis: θ/2θ, and
Scan range: 3 to 90°.

According to some aspects of the present invention, a heat absorption peak can be measured using DSC220U (Seiko Instruments Inc.) under the following conditions:
Temperature increase rate: 10° C./min,
Atmosphere: nitrogen, and
Measurement temperature range: 30 to 400° C.

In some aspects, the URAT1 inhibitor used in the present invention can be a class of chemical compounds represented by Formula (I) below, or a pure stereoisomer, pharmaceutically acceptable salt, hydrate, or solvate thereof.

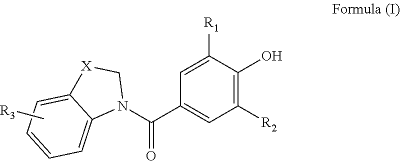

In certain aspects, each R₁ and R₂ is independently a lower alkyl group, a lower alkenyl group, a lower alkynyl group, a lower alkoxy group, a haloalkyl group, a haloalkoxy group, an alkylsulfanyl group, an alkylsulfinyl group, an alkylsulfonyl group, a lower alkyl-substituted carbamoyl group, a saturated nitrogen-containing heterocyclic N-carbonyl group, a halogen atom, a cyano group or a hydrogen atom.

In some aspects, R₃ is a lower alkyl group, a haloalkyl group, a halogen atom, a hydroxyl group or a hydrogen atom.

In other aspects, X represents a sulfur atom, —S(═O)— or —S(═O)₂—.

Xanthine oxidase (XO, or XAO) is a form of xanthine oxidoreductase that generates reactive oxygen species. XOs catalyze the oxidation of hypoxanthine to xanthine and can further catalyze the oxidation of xanthine to uric acid. These enzymes play an important role in the catabolism of purines in some species, including humans.

Disclosed herein is the use of a xanthine oxidase inhibitor (XOI) in combination with a URAT1 inhibitor and optionally with a SGLT2 inhibitor. As used herein, the term “xanthine oxidase inhibitor” refers to any substance that inhibits the activity of xanthine oxidase, an enzyme involved in purine metabolism. In humans, inhibition of xanthine oxidase reduces the production of uric acid, and several medications that inhibit xanthine oxidase are indicated for treatment of hyperuricemia and related medical conditions including gout. The XO inhibitor can be a purine analog such as allopurinol, oxypurinol, or tisopurine, or another molecule, such as febuxostat, topiroxostat or inositols (phytic acid and myo-inositol).

In some aspects, the XO inhibitor is selected from allopurinol, oxypurinol, tisopurine, febuxostat, topiroxostat or an inositol. In various aspects, the XO inhibitor is allopurinol or febuxostat.

The sodium/glucose cotransporter 2 (SGLT2) is a protein that in humans is encoded by the SLC5A2 (solute carrier family 5 (sodium/glucose cotransporter)) gene. SGLT2 is a member of the sodium glucose cotransporter family which are sodium-dependent glucose transport proteins. SGLT2 is the major cotransporter involved in glucose reabsorption in the kidney. SGLT2 is located in the early proximal tubule and is responsible for reabsorption of 80-90% of the glucose filtered by the kidney glomerulus. Most of the remaining glucose absorption is by sodium/glucose cotransporter 1 (SGLT1) in more distal sections of the proximal tubule. In some instances, inhibiting SGLT2 causes glycosuria. Inhibiting SGLT2 causes glycosuria-induced alteration of uric acid transport activity in renal tubules, therefore, SGLT2 inhibitors can lead to reduction in serum uric acid levels.

Disclosed herein is the use of a SGLT2 inhibitor in combination with a URAT1 inhibitor and optionally with a XO inhibitor. As used herein, the term “SGLT2 inhibitor” or “gliflozin” refers to a class of medications that alter essential physiology of the nephron; unlike SGLT1 inhibitors that modulate sodium/glucose channels in the intestinal mucosa. SGLT2 inhibitors are usually used in the treatment of type II diabetes mellitus (T2DM). Apart from blood sugar control, gliflozins have been shown to provide significant cardiovascular benefit in T2DM patients. Non-limiting examples of gliflozin include canagliflozin, dapagliflozin, empagliflozin, ertugliflozin, ipragliflozin, luseogliflozin, remogliflozin, sergliflozin etabonate, sotagliflozin (a dual SGLT1/SGLT2 inhibitor) and tofogliflozin.

The pharmaceutical compositions described herein can including a URAT1 inhibitor such as dotinurad alone or in combination with a SGLT2 inhibitor and/or a XO inhibitor. For example, the pharmaceutical composition can include dotinurad alone; dotinurad and a SGLT2 inhibitor; dotinurad and a XO inhibitor; or dotinurad, a XO inhibitor and a SGLT2 inhibitor.

By dotinurad, SGLT2 inhibitor and XO inhibitor, it is meant that the pharmaceutical composition can include dotinurad, SGLT2 inhibitor, XO inhibitor, or any pharmaceutically acceptable salt thereof. For example, the pharmaceutical composition can include dotinurad alone or a pharmaceutically acceptable salt thereof alone; dotinurad or a pharmaceutically acceptable salt thereof and a SGLT2 inhibitor or a pharmaceutically acceptable salt thereof; dotinurad or a pharmaceutically acceptable salt thereof and a XO inhibitor or a pharmaceutically acceptable salt thereof; or dotinurad or a pharmaceutically acceptable salt thereof, a XO inhibitor or a pharmaceutically acceptable salt thereof and a SGLT2 inhibitor or a pharmaceutically acceptable salt thereof.

The term “pharmaceutically acceptable salt” refers to physiologically and pharmaceutically acceptable salts of the compounds of the invention, e.g., salts that retain the desired biological activity of the parent compound and do not impart undesired toxicological effects thereto.

The pharmaceutical compositions described herein can including a URAT1 inhibitor alone or in combination with a SGLT2 inhibitor and/or a XO inhibitor and a pharmaceutical carrier.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

In one aspect, the composition is formulated in an oral dosage form. In various aspects, the oral dosage form is a tablet or a capsule.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

A tablet or capsule can include each active agent (URAT1 inhibitor, XO inhibitor and SGTL2 inhibitor) individually or can include any combination thereof. For example, one tablet or capsule can include a single active agent, and tablets/capsules can be administered for the delivery of one or more active agents to a subject (each active agent is administered separately). Alternatively, a single tablet or capsule can include a combination of the active agents to be administered to the subject for the simultaneous delivery of the combination of active agents (the combination of active agents is administered in a same tablet or capsule-fixed dose).

The combination of two or more active agents in a single tablet or capsule can provide synergic efficacy of the agents, improved reduction of serum uric acid and therefor, improved treatment of target diseases or conditions, as compared to each active agent used individually. In one aspect, the composition is formulated in a fixed-dose combination.

The pharmaceutical composition described herein can include 0.1-20 mg of dotinurad. For example, the pharmaceutical composition can include 0.1, 0.2, 0.3, 0.4, 0.5, 0.6, 0.7, 0.8, 0.9, 1, 1.5, 2, 3, 4, 5, 6, 7, 8, 9, 10, 12.5, 15, 17.5, 20, 22.5, 25, 30 or more mg of dotinurad, whether as a single dose or a fixed-dose in combination.

The pharmaceutical composition described herein can include 100-800 mg allopurinol. For example, the pharmaceutical composition can include 80, 90, 100, 110, 120, 130, 140, 150, 175, 200, 250, 300, 350, 400, 450, 500, 550, 600, 650, 700, 750, 800, 850, 900 or more mg allopurinol as a single dose or a fixed-dose in combination.

The pharmaceutical composition described herein can include 10-200 mg febuxostat. For example, the pharmaceutical composition can include 2.5, 5, 7.5, 10, 12.5, 15, 20, 30, 40, 50, 60, 70, 80, 90, 100, 110, 120, 130, 140, 150, 160, 170, 180, 190, 200, 210, 220 or more febuxostat in a single dose or a fixed-dose in combination.

In some aspects, the tablet or capsule includes 0.1-20 mg of dotinurad. In one aspect, allopurinol is present at 100-800 mg. In another aspect, febuxostat is present at 10-200 mg.

The pharmaceutical composition described herein can include 1-100 mg of SGLT2 inhibitor. For example, the pharmaceutical composition can include 1-10 mg or more SGLT2 inhibitor in a single dose or a fixed-dose in combination.

A tablet or capsule described herein can include a fixed-dose composition with a particular ratio of URAT inhibitor:XO inhibitor. For example, a tablet or capsule can include a ratio of dotinurad:allopurinol ranging from about 1:10 to 1:8000. For example, a ratio of dotinurad:allopurinol can be 1:10, 1:30, 1:50, 1:100, 1:250, 1:500, 1:750, 1:1000, 1:2000, 1:3000, 1:4000, 1:5000, 1:6000, 1:7000, 1:8000 or more.

A tablet or capsule can include a ratio of dotinurad:febuxostat ranging from about 3:1 to 1:2000. For example, a ratio of dotinurad:febuxostat can be 3:1, 2:1, 1:1, 1:5, 1:10, 1:25, 1:50, 1:100, 1:250, 1:500, 1:750, 1:1000, 1:1250, 1:1500, 1:1750, 1:2000, 1:2250, 1:2500 or more.

In some aspect, a tablet or capsule includes 0.1-20 mg of dotinurad and 100-800 mg allopurinol. In other aspects, a tablet or capsule includes 0.1-20 mg of dotinurad and 10-200 mg febuxostat.

In one aspect, the composition is for the treatment of chronic kidney disease (CKD).

Chronic kidney disease (CKD) is a kidney condition characterized by a gradual loss of kidney function over time (from months to years). It is estimated that chronic kidney disease affected 753 million people globally in 2016: 417 million females and 336 million males. In 2015 it caused 1.2 million deaths, up from 409,000 in 1990. The causes that contribute to the greatest number of deaths are high blood pressure at 550,000, followed by diabetes at 418,000, and glomerulonephritis at 238,000. Initially there are generally no symptoms; later, symptoms may include leg swelling, feeling tired, vomiting, loss of appetite, and confusion. Complications include an increased risk of heart disease, high blood pressure, bone disease, and anemia. CKD does not initially present any symptoms and is usually detected on routine screening blood work by either an increase in serum creatinine, or protein in the urine. As the kidney function decreases and additional symptoms appear including increased blood pressure (due to fluid overload and production of vasoactive hormones created by the kidney via the renin-angiotensin system, increasing the risk of developing hypertension and heart failure), urea accumulation (leading to azotemia and ultimately uremia), potassium accumulation in the blood (hyperkalemia with a range of symptoms including malaise and potentially fatal cardiac arrhythmias), fluid overload symptoms (may range from mild edema to life-threatening pulmonary edema), hyperphosphatemia (resulting from poor phosphate elimination in the kidney), hypocalcemia (resulting from 1,25 dihydroxyvitamin D3 deficiency, changes in mineral and bone metabolism (which may cause 1) abnormalities of calcium, phosphorus (phosphate), parathyroid hormone, or vitamin D metabolism; 2) abnormalities in bone turnover, mineralization, volume, linear growth, or strength (kidney osteodystrophy); and 3) vascular or other soft-tissue calcification), metabolic acidosis (may result from decreased capacity to generate enough ammonia from the cells of the proximal tubule), and anemia. In later stages, cachexia may develop, leading to unintentional weight loss, muscle wasting, weakness and anorexia. Sexual dysfunction is also very common in both men and women with CKD.

Causes of chronic kidney disease include diabetes, high blood pressure, glomerulonephritis, and polycystic kidney disease. Risk factors include a family history of chronic kidney disease. Diagnosis is by blood tests to measure the estimated glomerular filtration rate (eGFR), and a urine test to measure albumin. Ultrasound or kidney biopsy may be performed to determine the underlying cause.

Screening at-risk people is recommended to initial treatments and attempts to prevent disease progression. Initial treatment includes medications to lower blood pressure, blood sugar, and cholesterol. For example, angiotensin converting enzyme inhibitors (ACEIs) or angiotensin II receptor antagonists (ARBs) are generally first-line agents for blood pressure control, as they slow progression of the kidney disease and the risk of heart disease. Loop diuretics may be used to control edema and, if needed, to further lower blood pressure. Other recommended measures include staying active, and certain dietary changes such as a low-salt diet and the right amount of protein. Treatments for anemia and bone disease may also be required. Severe disease requires hemodialysis, peritoneal dialysis, or a kidney transplant for survival.

In another embodiment, the invention provides a method of treating CKD in a subject including administering to the subject a therapeutically effective amount of any of the pharmaceutical compositions described herein or a composition including a URAT1 inhibitor.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Therefore, the methods of treatment of a disease or condition, as described herein, also include methods of prevention of the disease or condition. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the CKD). Such amount should be sufficient for example to cure, slow down, lessen albuminuria, proteinuria, elevated creatinine clearance, elevated glomerular filtration rate, high blood pressure, type 2 diabetes, or a combination thereof. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. In various aspects, the administration is oral.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In some aspects, administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment. Specifically, the administration of dotinurad, to a subject can be in combination with allopurinol and/or a SGTL2 inhibitor. Such therapies can be administered prior to, simultaneously with, or following administration of the composition of the present invention.

In various aspects, the URAT1 inhibitor (e.g., dotinurad), XO inhibitor and/or SGTL2 inhibitor are administered simultaneously.

In one aspect, the CKD is characterized by albuminuria, proteinuria, elevated creatinine clearance, hyperuricemia, inflammatory infiltration, tubular damage, fibrosis, reduced glomerular filtration rate, high blood pressure, type 2 diabetes, or a combination thereof.

In an additional embodiment, the invention provides a method of treating non-alcoholic steatohepatitis (NASH) in a subject including administering to the subject a therapeutically effective amount of any of the pharmaceutical compositions described herein or a composition including a URAT1 inhibitor.

Non-alcoholic fatty liver disease (NAFLD), also known as metabolic (dysfunction) associated fatty liver disease (MAFLD), is excessive fat build-up in the liver without another clear cause such as alcohol use. There are two types non-alcoholic fatty liver (NAFL) and non-alcoholic steatohepatitis (NASH), with the latter also including liver inflammation. Non-alcoholic fatty liver disease is less dangerous than NASH and usually does not progress to NASH or liver cirrhosis. When NAFLD does progress to NASH, it may eventually lead to complications such as cirrhosis, liver cancer, liver failure, cardiovascular disease. Obesity and type 2 diabetes are strong risk factors for NAFLD. Other risks include being overweight, metabolic syndrome (defined as at least three of the five following medical conditions: abdominal obesity, high blood pressure, high blood sugar, high serum triglycerides, and low serum HDL cholesterol), a diet high in fructose, and older age. NAFLD and alcoholic liver disease are types of fatty liver disease. Obtaining a sample of the liver after excluding other potential causes of fatty liver can confirm the diagnosis.

Weight loss is the most effective treatment for NAFLD. A loss of 4% to 10% body weight is recommended, with 10% to 40% weight loss completely reversing NASH without cirrhosis. Other treatments including medications are primarily aimed at improving liver disease and is generally limited to those with biopsy-proven NASH and fibrosis. While many treatments appear to improve biochemical markers such as alanine transaminase levels, most do not reverse histological abnormalities or improve outcomes. For example, statin medications appear to improve liver histology and markers of liver biochemistry in people with NAFLD. Since people with NAFLD are at a higher risk of cardiovascular disease, statin treatment is indicated. People with NAFLD are not at higher risk for serious liver injury from statins. However, even if statins are safe to use in people with NASH cirrhosis, they should be avoided in people with decompensated cirrhosis.

In a further embodiment, the invention provides a method of treating heart failure in a subject including administering to the subject a therapeutically effective amount of any of the pharmaceutical compositions described herein or a composition including a URAT1 inhibitor.

Heart failure (HF), also known as congestive heart failure (CHF), (congestive) cardiac failure (CCF), and decompensation cordis, is a condition characterized by the inability of the heart to pump sufficiently to maintain blood flow to meet the body tissues' needs for metabolism. Signs and symptoms of heart failure commonly include shortness of breath, excessive tiredness, and leg swelling. The shortness of breath is usually worse with exercise or while lying down and may wake the person at night. A limited ability to exercise is also a common feature. Chest pain, including angina, does not typically occur due to heart failure. Common causes of heart failure include coronary artery disease, including a previous myocardial infarction (heart attack), high blood pressure, atrial fibrillation, valvular heart disease, excess alcohol use, infection, and cardiomyopathy of an unknown cause. These cause heart failure by changing either the structure or the function of the heart.

Treatment of HF depends on the severity and cause of the disease. In people with chronic stable mild heart failure, treatment commonly consists of lifestyle modifications such as stopping smoking, physical exercise, and dietary changes, as well as medications. In those with heart failure due to left ventricular dysfunction, angiotensin converting enzyme inhibitors, angiotensin receptor blockers, or valsartan/sacubitril along with beta blockers are recommended. For those with severe disease, aldosterone antagonists, or hydralazine with a nitrate may be used. Diuretics are useful for preventing fluid retention and the resulting shortness of breath. Sometimes, depending on the cause, an implanted device such as a pacemaker or an implantable cardiac defibrillator may be recommended.

In one embodiment, the present invention provides a method of treating gout and/or hyperuricemia in a subject including administering to the subject a therapeutically effective amount of any of the pharmaceutical compositions described herein or a composition including a URAT1 inhibitor.

Hyperuricemia is an abnormally high level of uric acid in the blood. In the pH conditions of body fluid, uric acid exists largely as urate, the ion form. Serum uric acid concentrations greater than 6 mg/dL for females, 7 mg/dL for men, and 5.5 mg/dL for youth (under 18 years old) are defined as hyperuricemia. The amount of urate in the body depends on the balance between the amount of purines eaten in food, the amount of urate synthesized within the body (e.g., through cell turnover), and the amount of urate that is excreted in urine or through the gastrointestinal tract. Hyperuricemia may be the result of increased production of uric acid, decreased excretion of uric acid, or both increased production and reduced excretion.

Many factors contribute to hyperuricemia, including genetics, insulin resistance, iron overload, hypertension, hypothyroidism, chronic kidney disease, obesity, diet, use of diuretics (e.g. thiazides, loop diuretics), and excessive consumption of alcoholic beverages.

Hyperuricemia experienced as gout is a common complication of solid organ transplant. Apart from normal variation (with a genetic component), tumor lysis syndrome produces extreme levels of uric acid, mainly leading to kidney failure. The Lesch-Nyhan syndrome is also associated with extremely high levels of uric acid.

Gout is a disorder of purine metabolism and occurs when its final metabolite, uric acid, crystallizes in the form of monosodium urate, precipitating and forming deposits (tophi) in joints, on tendons, and in the surrounding tissues. Microscopic tophi may be walled off by a ring of proteins, which blocks interaction of the crystals with cells and therefore avoids inflammation. Naked crystals may break out of walled-off tophi due to minor physical damage to the joint, medical or surgical stress, or rapid changes in uric acid levels. When they break through the tophi, they trigger a local immune-mediated inflammatory reaction in macrophages, which is initiated by the NLRP3 inflammasome protein complex. An evolutionary loss of urate oxidase (uricase), which breaks down uric acid, in humans and higher primates has made this condition common. Gout is a form of inflammatory arthritis characterized by recurrent attacks of a red, tender, hot, and swollen joint. Pain typically comes on rapidly, reaching maximal intensity in less than 12 hours. The joint at the base of the big toe is affected in about half of cases. It may also result in tophi, kidney stones, or kidney damage.

Gout is due to persistently elevated levels of uric acid in the blood, which occurs from a combination of diet, other health problems, and genetic factors. At high levels, uric acid crystallizes and the crystals deposit in joints, tendons, and surrounding tissues, resulting in an attack of gout. Gout occurs more commonly in those who regularly drink beer or sugar-sweetened beverages or who eat foods that are high in purines such as liver, shellfish, or anchovies, or are overweight. Diagnosis of gout may be confirmed by the presence of crystals in the joint fluid or in a deposit outside the joint.

Treatment with nonsteroidal anti-inflammatory drugs (NSAIDs), glucocorticoids, or colchicine improves symptoms. Once the acute attack subsides, levels of uric acid can be lowered via lifestyle changes and in those with frequent attacks, allopurinol or probenecid provides long-term prevention. Taking vitamin C and eating a diet high in low-fat dairy products may be preventive. Subject with gout who are intolerant to other medications can be administered pegloticase as an intravenous infusion every two weeks to reduce uric acid levels in this population. Pegloticase is a medication for the treatment of severe, treatment-refractory, chronic gout. It is a third line treatment in those in whom other treatments are not tolerated to help lower uric acid levels and reduced deposits of uric acid crystals in joints and soft tissue.

In one aspect, an effective amount of pegloticase is further administered to the subject.

Presented below are examples discussing the use of a URAT1 inhibitor (e.g., dotinurad) in combination with a XO inhibitor and/or a SGLT2 inhibitor contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Evaluation of the Effects of Dotinurad in Combination with Allopurinol on CKD

To evaluate the effects of dotinurad and allopurinol on CKD, the adenine-induce CKD model or the 5/6 nephrectomy model of CKD can be used.

The 5/6 nephrectomy (5/6 Nx) model of CKD is a widely used model of CKD that resembles stages 3-4 of CKD in humans. The model is approached by right nephrectomy and surgical resection of left kidney poles (CKD-P) or ligation of 2-3 branches of the left renal artery (CKD-I). The differences between these two approaches are related to greater activation of the renin-angiotensin-aldosterone system and faster development of systemic hypertension in the infarction model. Hypertension is the most common comorbidity in CKD patients; therefore, CKD-I is more related to the human condition.

5/6 Nx rats can be dosed with potassium oxonate (750 mg/kg BW) to increase serum uric acid. In the context of CKD, hyperuricemia aggravated the disease; therefore, rats had more proteinuria, arteriolopathy, tubulointerstitial inflammation and fibrosis, and CKD hyperuricemic rats developed profound renal vasoconstriction. Allopurinol and febuxostat prevented the aggravation of the disease. Febuxostat provided significant nephroprotection even in 5/6 rats that not received oxonic acid. (Sanchez-Lozada et al. Kidney Int. 2005:67(1):237-47; Sanchez-Lozada et al. Nephron Physiol. 2008; 108(4):69-78)

The adenine model is very versatile as renal damage can be modulated by the dose given. Another advantage is that this model provides more biological material to work with than the 5/6 Nx model. On the other hand, in the 5/6 Nx model, a significant proportion of renal mass is excised at once; therefore, renal disease progression is less related to the human condition, although this model is still widely used to study the mechanisms of chronic renal disease and progression.

Dotinurad is a highly specific human URAT1 blocker. Renal urate transporter renal-specific (RST) is the murine orthologue of hURAT1. These two transporters are 74% identical and 81% similar at the amino acid level; therefore, one could expect a reduced urate-lowering response to an equivalent dose/weight of dotinurad in rats in comparison to humans. In addition, murine xanthine oxidase is 100 times more active than humans, and this also contributes to a lower response to uricosurics in these species as such effect was sensitive to the rate of urate synthesis in rats. Thus, to overcome this effect, hyperurcemic rats have been treated with low doses of a xanthine oxidase inhibitor (topiroxostat, 0.1 mg/kg BW). It was shown that slowing urate synthesis rate provides a useful model for evaluating uricosuric agents, including dotinurad, an hURAT-1 inhibitor.

In order to assess the effects of dotinurad on CKD, two approaches can be implemented. In a prevention approach dotinurad is started simultaneously with the CKD induction. In the treatment approach, dotinurad administration is delayed until CKD stabilizes. In this approach, dotinurad treatment is started 4 weeks after CKD initiation.

### Methods:

Regardless of the approach followed, the experiments will be performed as follow:

### Blood Pressure:

Blood pressure will be measured in conscious rats by tail-cuff sphygmomanometer.

### Plasma Analysis:

Plasma urea nitrogen, creatinine, uric acid, will be measured using commercial kits. HMGB1 will be measured with an ELISA kit.

### Urine Analysis:

Urine proteins will be measured by Bradford method. Creatinine and uric acid will be measured using commercial kits. Urate fractional excretion will be calculated.

### Glomerular Filtration Rate:

Glomerular filtration rate will be assessed with a transdermic monitor using FITC-Sinistrin, as a marker of GFR, injected via the rat tail in 3 rats per/group.

### Histology:

Fixed renal tissue will be embedded in paraffin, processed accordingly. Slides will be stained with Masson's trichromic or Syrius red to evaluate the degree of tubulointerstitial fibrosis. Slides stained with PAS will be used for IMHC against alpha-smooth muscle actin and E-Cadherin. Renal epithelial-mesenchymal transition will be evaluated by the expression of E-cadherin and alpha-smooth muscle actin in proximal tubules. Aortas will be peeled off and sectioned in 4 mm segments that will be fixed in 10% buffered formalin and stained with Von Kossa for calcium deposition analysis. Slides will be analyzed by light microscopy (Olympus BX51) and captured by a digital camera (Vr-Evolution, Media Cybernetics). The images will be processed using Image-Pro-Plus, version 7.2 (Image-Pro INC, Media Cybernetics).

### Aorta Tissue Analysis:

Uric acid content will be measured. Calcium content will be measured using a colorimetric based kit (Abcam).

### Western Blotting:

Western blotting will be performed in renal tissue or aortic segments homogenates of at least three samples randomly chosen from each group. Proteins of interest in aorta (HMBG1 and URAT-1) and renal tissue (TGF-beta). Bands will be visualized using horseradish peroxidase (HRP) secondary antibodies and Immobilon Crescendo Western HRP Substrate (Merck Millipore, Billerina MA, USA). Immunoblots will be analyzed using Image Studio Lite 5.2 software (Licor Biosciences, Lincoln, NB, USA).

### Statistical Analysis:

Multiple group comparisons will be performed by using a one-way analysis of variance (ANOVA) with posttest according to Tukey's. A P value of <0.05 will be considered statistically significant.

### Prevention Approach Experimental Groups:

Male Wistar rats (200-250 g) will be divided into 5 groups:
1-P) Normal control,
2-P) CKD
3-P) CKD+Dotinurad (dose to define. Dotinurad was used at 30-100 mg/kg BW acutely in rats),
4-P) CKD+Dotinurad+Allopurinol (1 mg/kg BW)
5-P) CKD+Allopurinol

A minimum of seven rats for each experimental group will be studied. Groups will be followed for 8-9 weeks. At 4- and 8-weeks rats will be collected for urine in metabolic cages, systolic blood pressure and glomerular filtration rate (GFR) will be measured. Finally, rats will be euthanized, and blood, kidney tissue, and aorta collected for further analysis.

### Treatment Approach Experimental Groups:

Male Wistar rats (200-250 g) will be divided into 5 groups:
1-T) Normal control,
2-T) CKD, 4 wks follow-up (this group will allow to study the renal and CV alterations before starting the treatments)
2a-T) CKD, 8 wks follow-up
3-T) CKD+Dotinurad (dose to define. Dotinurad was used at 30 mg/kg BW acutely in rats (29)),
4-T) CKD+Dotinurad+Allopurinol (1 mg/kg BW).
5-T) CKD+Allopurinol

A minimum of seven rats for each experimental group will be studied. In this case both dotinurad and allopurinol dosing will start after 4 weeks of CKD. A similar renal disease progression will be assessed at 2 wks using one of the following criteria: SBP>140 mmHg, BUN=30-70 mg/dL, or Uprot>30 mg/24 h. At 4 wks, rats will have urine collected in metabolic cages, and systolic blood pressure will be measured. Rats with a systolic blood pressure>160 mmHg and Uprot>40 mg/d will be followed for the additional 4-5 weeks. At 8-weeks, rats will have urine collected again in metabolic cages, and systolic blood pressure and GFR will be measured. Finally, rats will be euthanized, and blood, kidney tissue, and aorta collected for further analysis.

### Measurements:

In all groups, baseline measurements of body weight, systolic blood pressure, proteinuria, plasma creatinine, and BUN will be performed. Rats will be staggered allocated 1/group (5 rats in total per day) to control the experimental groups sampling better.

However, there is evidence that both high mobility group box 1 (HMGB1) and beta-catenin induce aortic vascular calcification. Interestingly, uric acid is associated with the activation of those pathways.

### Outcomes:

Bodyweight, renal function (BUN, serum creatinine, GFR), serum uric acid, HMGB1; urine creatinine, uric acid, and protein excretion. In renal tissue fibrosis, arteriolopathy and markers of epithelial-to-mesenchymal transition (EMT) will be evaluated. A segment will be fixed for histological analysis (calcium deposits) in the aorta, and another segment will be snap-frozen and stored in liquid nitrogen for calcium and UA content and expression of HMBG1 and URAT-1 by western-blot.

Although the invention has been described with reference to the above examples, it will be understood that modifications and variations are encompassed within the spirit and scope of the invention. Accordingly, the invention is limited only by the following claims.

1. A pharmaceutical composition comprising a URAT1 inhibitor alone or in combination with a sodium-glucose cotransporter-2 (SGLT2) inhibitor and/or a xanthine oxidase (XO) inhibitor and a pharmaceutical carrier.

2. The composition of claim 1, for the treatment of chronic kidney disease (CKD).

3. The composition of claim 1, wherein the URAT1 inhibitor is dotinurad, a pharmaceutically acceptable salt, hydrate, or solvate thereof.

4. The composition of claim 3, wherein dotinurad is an amorphous form.

5. The composition of claim 3, wherein dotinurad is a type II crystal having characteristic peaks at least about 15.1, 18.1, 22.8, 23.7 and 24.0 degrees in a diffraction angle (2θ) by X-ray powder diffraction.

6. The composition of claim 5, wherein the type II dotinurad crystal has a heat absorption peak at about 212° C. in differential scanning calorimetry (DSC) analysis.

7. The composition of claim 3, wherein dotinurad is a type I crystal having characteristic peaks at least about 11.5, 14.6, 18.2, 24.0 and 25.5 degrees in a diffraction angle (2θ) by X-ray powder diffraction.

8. The composition of claim 7, wherein the type I dotinurad crystal has a heat absorption peak at about 191° C. in DSC analysis.

9. The composition of claim 3, formulated in an oral dosage form.

10. The composition of claim 9, wherein the oral dosage form is a tablet or a capsule.

11. The composition of claim 10, wherein the tablet or capsule comprises 0.1-20 mg of dotinurad.

12. The composition of claim 1, wherein the SGLT2 inhibitor is selected from canagliflozin, dapagliflozin, empagliflozin, ertugliflozin, ipraglifolozin, luseogliflozin, remoglifolozin, sergliflozin, sotagliflozin, or tofogliflozin.

13. The composition of claim 1, wherein the XO inhibitor is selected from allopurinol, oxypurinol, tisopurine, febuxostat, topiroxostat or an inositol.

14. The composition of claim 1, formulated in a fixed dose combination.

15. The composition of claim 13, wherein the XO inhibitor is allopurinol or febuxostat.

16. The composition of claim 15, wherein allopurinol is present at 100-800 mg.

17. The composition of claim 15, wherein the febuxostat is present at 10-200 mg.

18. A method of treating chronic kidney disease (CKD) in a subject comprising administering to the subject a therapeutically effective amount of a pharmaceutical composition of claim 1 or a composition comprising a URAT1 inhibitor.

19. The method of claim 18, wherein the URAT1 inhibitor is dotinurad.

20. (canceled)

21. A method of treating non-alcoholic fatty liver disease (NAFLD) and/or non-alcoholic steatohepatitis (NASH) in a subject comprising administering to the subject a therapeutically effective amount of a pharmaceutical composition of claim 1 or a composition comprising a URAT1 inhibitor.

22-24. (canceled)

25. A method of treating gout and/or hyperuricemia in a subject comprising administering to the subject a therapeutically effective amount of a pharmaceutical composition of claim 1 or a composition comprising a URAT1 inhibitor.

26-27. (canceled)
